(19) United States
(12) Patent Application Publication
(10) Pub. No.: US 2025/0010515 A1
(43) Pub. Date: Jan. 9, 2025

(54) METHOD AND APPARATUS FOR HIGH-THROUGHPUT PREPARATION OF A CEMENT-BASED MATERIAL

(71) Applicants: CHINA BUILDING MATERIALS ACADEMY, Beijing (CN); CHINA NATIONAL BUILDING MATERIAL GROUP CO., LTD., Beijing (CN)

(72) Inventors: Xuehong REN, Beijing (CN); Jiayuan YE, Beijing (CN); Xiao ZHI, Beijing (CN); Hongtao ZHANG, Beijing (CN); Di SHI, Beijing (CN); Wensheng ZHANG, Beijing (CN); Zhaijun WEN, Beijing (CN)

(21) Appl. No.: 18/546,230

(22) PCT Filed: Mar. 1, 2023
(86) PCT No.: PCT/CN2023/079051

§ 371 Date: Aug. 11, 2023

(30) Foreign Application Priority Data
Aug. 31, 2022 (CN) 202211062709.5

(51) Int. Cl. B28C 7/00; B28C 9/00; C04B 7/36; G01N 33/38

(52) U.S. Cl. CPC B28C 7/0007; B28C 9/002; C04B 7/36; G01N 33/383

(57) ABSTRACT

A method and an apparatus for high-throughput preparation of a cement-based material are released and belong to the technical field of cement production. According to the technical solution of the present application, a single-mine, a single-phase or a unit components maintaining specific hydration hardening characteristics are used as structural units, and the method comprises the following steps that (1) the structural units are placed in storage tubes X1, X2, X3 . . . Xn respectively; (2) the materials in the storage pipes are put according to the composition design proportion of the cement-based material, and Y1, Y2, Y3 . . . Ym of mixed materials are prepared; (3) fully and uniformly mixing the Y1, Y2, Y3 . . . Ym of mixed materials through a uniform mixing device; and 4) respectively filling the Y1, Y2, Y3 . . . Ym of uniformly mixed materials into storage tanks Z1, Z2, Z3 . . . Zm to prepare m groups of cement-based material samples. The method and device are easy to operate and high in applicability, and the material research and development manpower and resource cost can be greatly reduced.

### CROSS-REFERENCE TO RELATED APPLICATIONS

The present application claims priority benefits to Chinese Patent Application No. 202211062709.5, filed on Aug. 31, 2022, titled “METHOD AND APPARATUS FOR HIGH-THROUGHPUT PREPARATION OF CEMENT-BASED MATERIAL”, the contents of which are incorporated herein by reference.

### TECHNICAL FIELD

The present application belongs to the technical field of cement production, and in particular relates to a method and apparatus for high-throughput preparation of a cement-based material.

### BACKGROUND

Since the “Material Genome Project” was proposed by the United States in 2011, European Union, Japan, Russia, and China have also successively released material genome research projects, which vigorously promote the transformation of material research and development concepts. As the most basic and bulky traditional cement-based materials, cement and concrete play a key and irreplaceable role in national infrastructure constructions, major engineering constructions, and national defence projects. The traditional R&D methods for cement-based materials are mainly based on the “trial and error method”, which is time-consuming and laborious, and easily cause huge consumption of human, material, and financial costs. That no longer meets the development needs of China's modernization construction. Therefore, in order to quickly screen and effectively improve the performance of cement-based materials and save R&D time and economic costs, there is an urgent need to transform the traditional R&D paradigm of cement-based materials and develop high-throughput experimental technologies for cement-based materials based on material genetic engineering. This is an inevitable trend for material technology innovation and progress, and also a fundamental requirement for efficient and intelligent engineering constructions in the future.

Material genes are generally understood as basic structural units that maintain the intrinsic properties of materials. However, cement-based materials are complex multi-component and multi-phase systems, and their preparation process is a complex and typical multi-sequence process. Cement-based material products comprise different intermediate and terminal products such as cement clinker, cement, concrete and so on. Each stage involves different raw materials, numbers, and types, and is accompanied by complex physical and chemical reaction processes and processing techniques. At present, the research on high-throughput preparation technology for cement-based materials is still blank at home and abroad. This also leads to the fact that the research methods for cement-based materials are still in the traditional “trial and error” stage, with low efficiency in preparation and optimization, and long R&D cycles.

Therefore, how to develop a rapid, low-cost, and efficient method for high-throughput preparation of a cement-based material based on the concept of material genetic engineering is the foundation and prerequisite for achieving rapid iterative optimization of cement-based materials. The application of this technology will contribute to the transformation of the R&D paradigm of cement-based materials, effectively promoting the rapid development and engineering application of cement-based materials with better performance, lower carbon content, and specific hydration and hardening properties, and assisting the rapid transformation and development of the cement industry towards high-quality, green, and low-carbon directions.

### SUMMARY

In response to the above-mentioned technical problems, the present application provides a method and apparatus for high-throughput preparation of a cement-based material that focuses on accelerating the development of cement-based materials and assisting in the transformation of the R&D paradigm of cement-based materials. The method of the present application takes single-mine, single-phase, or independent components that maintain specific hydration and hardening properties as the basic elements of the structural unit. Rapid screening and customization optimization of the material composition and performance can be achieved by measuring and analyzing the performance of the sample, optimizing and controlling the ratio, fineness, or gradation of the unit elements, thereby greatly shortening the R&D cycle and saving R&D costs. The present application also proposes an apparatus for high-throughput preparation of cement-based materials.

The technical solution of this present application is: single-mine, single-phase, or unit components maintaining specific hydration and hardening characteristics are used as structural units,

The technical solution is carried out as following steps:
1) placing the structural units in storage tubes X1, X2, X3 . . . Xn respectively;
2) putting the materials in each of the storage tubes according to the design proportion of the cement-based materials, and then preparing Y1, Y2, Y3 . . . Ym of mixed materials respectively;
3) fully and uniformly mixing the Y1, Y2, Y3 . . . Ym of the mixed materials through a uniform mixing device respectively;
4) filling the Y1, Y2, Y3 . . . Ym of the uniformly mixed materials into storage tanks Z1, Z2, Z3 . . . Zm respectively to prepare m groups of cement-based material samples.

The structural unit is a single-mine of cement clinker which comprises alite (a solid solution of tricalcium silicate C₃S), belite (a solid solution of dicalcium silicate C₂S), tricalcium aluminate (C₃A), calcium aluminate (CA), calcium dialuminate (CA₂), mayenite (C₁₂A₇), dicalcium ferrite (C₂F), tetracalcium aluminoferrite (C₄AF), hexacalcium aluminodiferrite (C₆AF₂), hexacalcium dialuminoferrite (C₆A₂F), ye'elimite (C₄A₃$), calcium fluoaluminate (C₁₁A₇·CaF₂), calcium chloroaluminate (C₁₁A₇·CaCl₂), barium calcium sulphoaluminate (C₃A₃$·BaO), strontium calcium aluminate (C₃A₃$·SrO), ternesite (C₅S₂$), tricalcium phosphate (C₃P), tetracalcium phosphate (C₄P), calcium phosphoaluminate (C₈A₄P), gehlenite (C₂AS), periclase (MgO), free gypsum (f-CaSO₄), and free calcium oxide (f-CaO).

The structural unit further comprises the cement component unit, which is gypsum, slag, volcanic ash, fly ash, silica fume, and limestone.

The structural unit further comprises the concrete structural unit that includes sand, stone, and concrete admixtures, as well as supplementary cementitious material such as steel slag and so on.

The composition, structural and performance tests on the m groups of cement-based material samples are performed respectively for screening specific or excellent performance cement-based materials including t chemical composition, mineral composition, density, fineness, hydration heat evolution, setting time, strength, composition and structure of hydration products, volume soundness, impermeability, freeze-thaw resistance, workability, sulfate resistant, chloride resistant, and wear resistance.

An apparatus for realizing the method for high-throughput preparation of a cement-based material in the present application comprises:
n of primary storage silos are used for accommodating raw materials of cement-based material composition unit, wherein flow valves are provided at the bottom ends of each primary storage silos, and
m of secondary pre-loaded tanks are used for receiving materials from any two or more of n of the primary storage silos, and mixing the materials uniformly;
wherein n of the primary storage silos are arranged on a primary storage silo delivery device in a rotating or linear sliding manner, and m of the secondary pre-loaded tanks are arranged on a secondary pre-loaded tank delivery device in a rotating or linear sliding manner.

A feeding control device is provided between the primary storage silo delivery device and the secondary pre-loaded tank delivery device.

The apparatus further comprises m of terminal storage tanks for receiving target material discharged from the secondary pre-loaded tanks.

The present application aims at the multiple complexities of cement-based materials, breaks through the traditional high-throughput test preparation method using chemical elements as genetic units, proposes a method and test apparatus for high-throughput test preparation of cement-based materials based on using single-mine, single-phase, or unit components maintaining specific hydration and hardening characteristics as structural units, and solves the problem of high-throughput test preparing complex multi-component cement-based materials, which has laid a foundation for the application and promotion of material genetic engineering technology in the field of cement-based materials, accelerating the transformation of the R&D paradigm of cement-based materials promoting the development of the cement discipline.

The method in the present application can prepare and screen the performance of cement-based materials rapidly, shorten the research and development cycle of cement-based new materials, assist the cement industry in rapid iterations updating towards high-performance, green, and low-carbon directions, and have high economic and environmental benefits. According to the requirements of different projects and construction environments, the method can quickly screen and prepare different types and components of cement-based new materials, which effectively ensures the efficiency and quality of project construction and has broad social and economic benefits.

The method of the present application can achieve the integrated preparation research of different intermediate or terminal cement-based materials such as clinker, cement, concrete and so on, which is conducive to promoting the systematic development of related research the life cycle performance evaluation of cement-based materials and assisting the low-carbon and sustainable development of cement-based materials with significant social and environmental benefits. The method and apparatus of the present application are simple to operate and highly applicable, and can significantly reduce the human and resource costs of material research and development.

### BRIEF DESCRIPTION OF THE DRAWINGS

FIG. 1 is a structural schematic diagram of the apparatus of the present application.

FIG. 2 is a diagram of quantitative analysis results of M1 sample in the embodiment of the present application;

FIG. 3 is a diagram of quantitative analysis result of M2 sample in the embodiment of the present application;

FIG. 4 is a diagram of quantitative analysis result of M3 sample in the embodiment of the present application:

FIG. 5 is hydration heat release curves of different clinker samples in the present application;

FIG. 6 is a histogram of the compressive strength of three groups of new low-carbon clinker in the present application: S1, S2, and S3.

### DETAILED DESCRIPTION

The present application uses single-mine, single-phase, or unit components maintaining specific hydration and hardening characteristics as structural units, achieving rapid batch production of cement-based materials through high-throughput testing apparatus. This method is fast, efficient, low-carbon, environmentally friendly, and economical.

The present application comprises a method for high-throughput preparation of a cement-based material, which uses a single-ore, a single-phase, or unit components maintaining specific hydration and hardening characteristics as structural units.

The method is carried out as following steps:
1) placing the structural units in storage tubes X1, X2, X3 . . . Xn respectively;
2) putting the materials in each of the storage tubes according to the design proportion of the cement-based materials, and then preparing Y1, Y2, Y3 . . . Ym of mixed materials respectively;
3) fully and uniformly mixing the Y1, Y2, Y3 . . . Ym of the mixed materials through a uniform mixing device respectively;
4) filling the Y1, Y2, Y3 . . . Ym of the uniformly mixed materials into storage tanks Z1, Z2, Z3 . . . Zm respectively to prepare m groups of cement-based material samples.

Further, the structural unit is a single-mine of cement clinker which comprises alite (a solid solution of tricalcium silicate C₃S), belite (a solid solution of dicalcium silicate C₂S), tricalcium aluminate (C₃A), calcium aluminate (CA), calcium dialuminate (CA₂), mayenite (C₁₂A₇), dicalcium ferrite (C₂F), tetracalcium aluminoferrite (C₄AF), hexacalcium aluminodiferrite (C₆AF₂), hexacalcium dialuminoferrite (C₆A₂F), ye'elimite (C₄A₃$), calcium fluoaluminate (C₁₁A₇·CaF₂), calcium chloroaluminate (C₁₁A₇·CaCl₂)), barium calcium sulphoaluminate (C₃A₃$·BaO), strontium calcium aluminate (C₃A₃$·SrO), ternesite (C₅S₂$), tricalcium phosphate (C₃P), tetracalcium phosphate (C₄P), calcium phosphoaluminate (C₈A₄P), gehlenite (C₂AS), periclase (MgO), free gypsum (f-CaSO₄), and free calcium oxide (f-CaO).

Under the concept of the present application, a person skilled in this field can use the characteristic of various mineralogical phases, listed in the following table as the structural units of the present application. Of course, it cannot be ruled out that in the future, a person skilled in this field can make corresponding selections or discards when new discoveries are discovered regarding the properties of various mineral types in the table with technological progress in this field.

Cement clinker minerals and their properties

| Mineral types | Properties |
|---|---|
| Tricalcium silicate (C₃S) | Hydration reaction fast in the early stage, the large amount of |
| | hydration heat release and the main contributor to Portland |
| | cement's strength in the early and middle stages. |
| Dicalcium silicate (C₂S) | Hydration reaction slow, low strength in the early stages, |
| | sustainable growth of strength in the later stage and the main |
| | contributors to Portland cement's strength in the later stage |
| Tricalcium aluminate(C₃A) | Hydration and hardening fastly, fully hydratable in 3 days, |
| | but low strength and losing strength in the later stage, the |
| | large amount of dry shrinkage |
| Calcium aluminate (CA) | Normal coagulation, hydration and hardening rapid, the large |
| | amount hydration heat release, and the main contributors to |
| | the strength of aluminate cement |
| Calcium dialuminate (CA₂) | Hydration reaction slow, low strength in the early stage, and s |
| | sustainable growth of strength in the later stage |
| Monocalcium | Chemically inert mineral, no hydration activity, but high |
| hexaaluminate (CA₆) | heat resistance |
| Mayenite (C₁₂A₇) | Fast coagulation, hydration and hardening extremely fast, but |
| | the strength not as high as that of CA |
| Dicalcium ferrite (C₂F) | Weak hydration activity |
| Tetracalcium aluminoferrite | Hydration reaction fast, the hydration heat release only lower |
| (C₄AF) | than that of C₃A, and high strength in both early and later |
| | stages |
| Hexacalcium | Hydration heat release rate lower than that of C₄AF, low |
| aluminodiferrite (C₆AF₂) | strength in the early stage but high strength in the later stage |
| Hexacalcium | higher strength in the early stage but lower strength in the |
| dialuminoferrite (C₆A₂F) | later stage than that of C₄AF |
| Ye'elimite (C₄A₃$) | High hydration activity, early strength and expansion, the |
| | main contributor to the strength of sulphoaluminate cement |
| | in the early stage |
| Calcium fluoaluminate | Hydration and hardening fast, and fast growth of strength in |
| (C₁₁A₇•CaF₂) | the early stage, but low strength overall |
| Calcium chloroaluminate | Hydration performance similar to that of calcium |
| (C₁₁A₇•CaCl₂) | fluoaluminate, hydration and hardening fast |
| Barium calcium | Hydration and hardening fast, the strength being significantly |
| sulphoaluminate | higher than that of Ye'elimite |
| (C₃A₃$•BaO) | |
| Strontium calcium | Fast hydration and hardening, the strength being also higher |
| aluminate (C₃A₃$•SrO) | than that of Ye'elimite |
| Ternesite (C₅S₂$) | Good potential hydration activity higher than that of |
| | dicalcium silicate and hydration fully in the presence of |
| | aluminum sources existing |
| Tricalcium phosphate (C₃P) | Hydration Slow at room temperature |
| Tetracalcium phosphate | Mainly used as bone cement |
| (C₄P) | |
| Calcium phosphoaluminate | Mainly providing strength in the middle and late stages |
| (C₈A₄P) | |
| Perovskite (CT) | Unique electromagnetic properties such as isomerization and |
| | electrocatalysis |
| Gehlenite (C₂AS) | Very low hydration activity |
| Periclase (MgO) | Not conducive to the soundness of Portland cement |
| Free gypsum (f-CaSO₄) | Guaranteeing the formation of ettringite in sulphoaluminate |
| | cement |
| Free calcium oxide (f-CaO) | Affecting the soundness of cement |

Further, the structural unit further comprises the cement component unit, which is gypsum, slag, volcanic ash, fly ash, silica fume, and limestone.

Further, the structural unit further comprises the concrete structural unit that includes sand, stone, and concrete admixtures, as well as supplementary cementitious material such as steel slag and so on.

The composition, structural and performance tests on the m groups of cement-based material samples are performed respectively for screening specific or excellent performance cement-based materials including chemical composition, mineral composition, density, fineness, hydration heat evolution, setting time, strength, composition and structure of hydration products, volume soundness, impermeability, freeze-thaw resistance, workability, sulfate resistant, chloride resistant, and wear resistance.

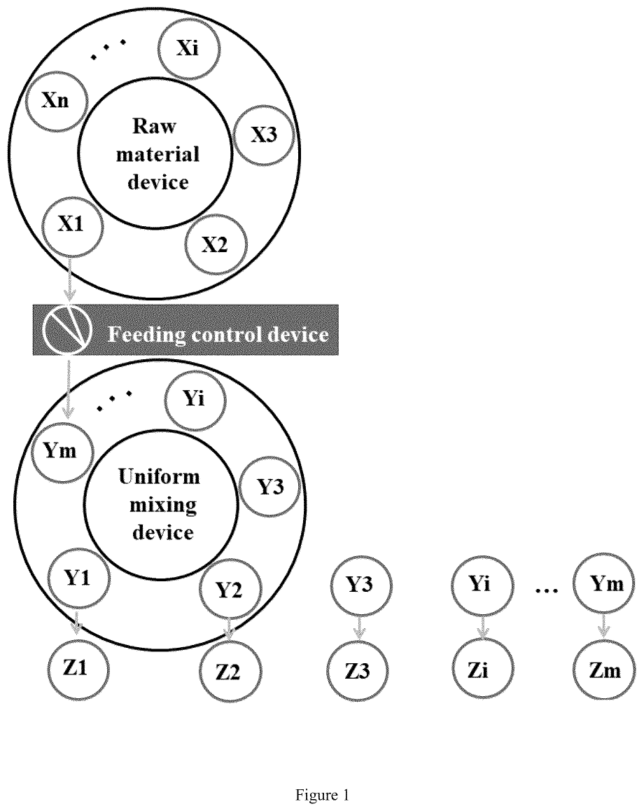

As shown in FIG. 1, an apparatus for realizing the method for high-throughput preparation of a cement-based material in the present application comprises: n of primary storage silos are used for accommodating raw materials of cement-based material composition unit, wherein flow valves are provided at the bottom ends of each primary storage silos,
and m of secondary pre-loaded tanks are used for receiving materials from any two or more of n of the primary storage silos, and mixing the materials uniformly;
wherein n of the primary storage silos are arranged on a primary storage silo delivery device in a rotating or linear sliding manner, and m of the secondary pre-loaded tanks are arranged on a secondary pre-loaded tank delivery device in a rotating or linear sliding manner. r;

A feeding control device is provided between the primary storage silo delivery device and the secondary pre-loaded tank delivery device.

The apparatus further comprises m of terminal storage tanks for receiving target material discharged from the secondary pre-loaded tanks.

The apparatus for realizing the method for high-throughput preparation of a cement-based material in the present application uses single-mine, single-phase, or unit components maintaining specific hydration and hardening characteristics as structural units, and the intrinsic properties of the materials can be effectively controlled. After storing them on a disk-shaped or linear movable device, the unit materials can be flexibly configured and mixed according to design requirements. Further, it can solve the problem of high-throughput experimental preparation of complex multielement cement-based materials, realize screening and optimization of material composition and performance rapidly, and greatly shorten the R&D cycle and save research and development costs.

In order to explain the present application in detail and better understand the technical solution and advantages of the present application, the following will provide a detailed description of the present application in combination with embodiments and drawings, but the present application is not limited to the following embodiments.

### Embodiment 1

The target of embodiment 1 is to prepare a Portland cement clinker with the 300±20 m²/kg surface area. The design proportion of the cement clinker to be prepared is shown in Table 1. Four cement clinker single-mine, i.e., Alite (solid solution of tricalcium silicate), Belite (solid solution of dicalcium silicate), tricalcium aluminate, and tetracalcium aluminoferrite, are calcined and prepared respectively according to the design of the target cement clinker. Four types of clinker are finely ground into powder with the 300±20m/kg surface area. The above-mentioned single-mine raw materials are placed in the corresponding unit material storage tubes X1, X2, X3, and X4 of the high-throughput test apparatus (see FIG. 1) respectively, wherein mixed zircon balls are added to storage tube X5. The raw material storage tube is placed into a raw material device in a circular arrangement. The raw material proportion of the cement clinker prepared is given according to Table 1, and the weight proportion of the raw material is controlled by controlling the valve size of the storage tube orifice through the feeding control device. Different types and weights of raw materials are placed into different target material pre-loaded tanks through 360-degree circularly rotating the raw material device. 200 g zircon balls are added to the pre-loaded tank for each. The pre-loaded tank is placed into the uniform mixing device. After the target clinker is evenly mixed in the pre-loaded tank, the zircon balls are separated through a mesh filter screen. The mixed cement clinker enters the target material terminal storage tanks Z1, Z2, and Z3 respectively, and the storage tanks are hermetically stored to obtain three sets of clinker samples, M1, M2, and M3.

TABLE 1
Composition of designed clinker (wt %)

| | | | Tricalcium | Tetracalcium |
|---|---|---|---|---|
| No. | Alite | Belite | aluminate | aluminoferrite |
| M1 | 65 | 20 | 3 | 12 |
| M2 | 58 | 25 | 10 | 7 |
| M3 | 65 | 18 | 5 | 12 |

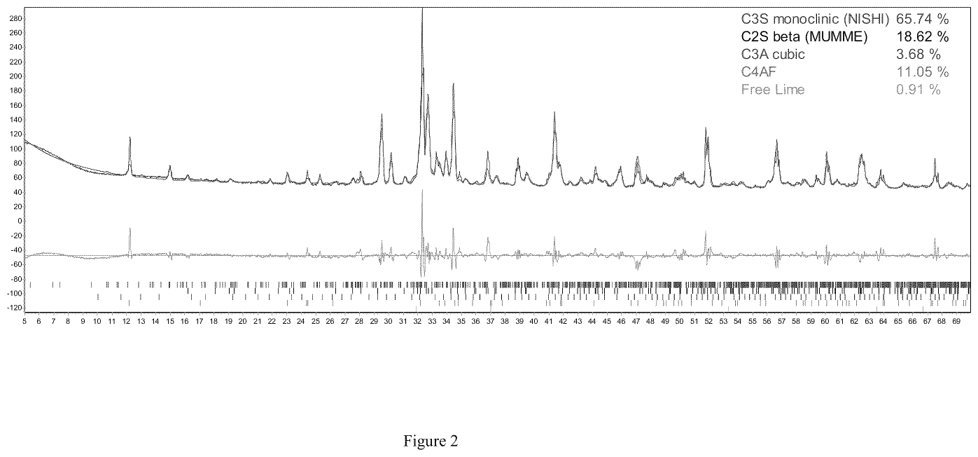

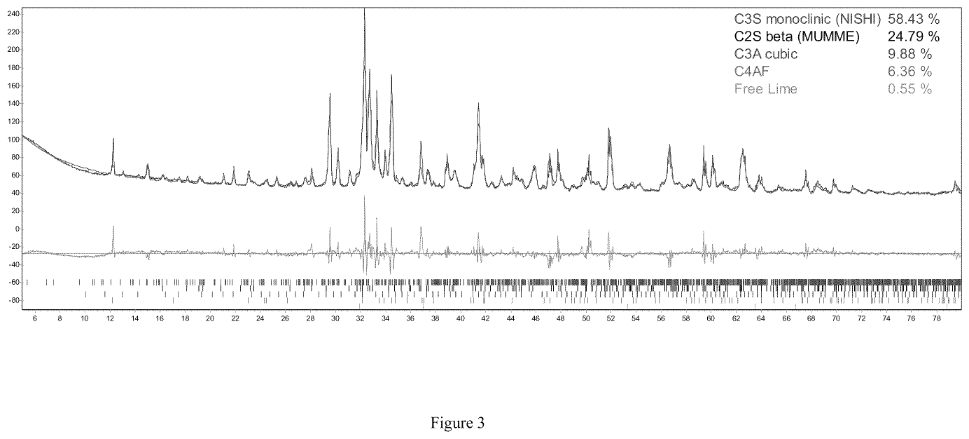

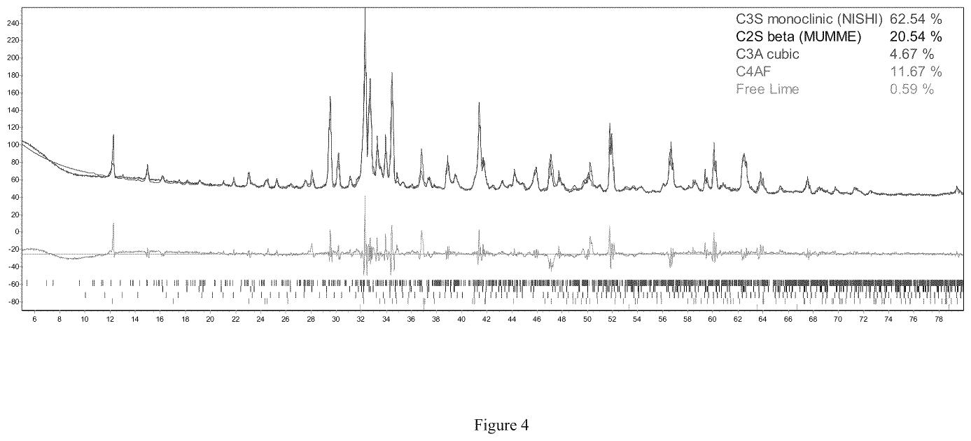

The mineral composition of the M1, M2, and M3 clinkers prepared above is measured by XRD (X-ray diffraction), and the XRD results are quantitatively analyzed by the Rietveld method. The results show that the refined fitting Rwp factor for different samples is far below 15, with an average of between 7 and 8. The refined fitting effect is shown in FIGS. 2 to 4. The XRD quantitative analysis results of the sample are shown in Table 2. It can be seen from Table 2 that the XRD determination results tally with the set composition of the clinker. This indicates that the cement clinker with the set composition has been successfully prepared according to this method.

TABLE 2
Composition of determined clinker (wt %)

| | | | Tricalcium | Tetracalcium |
|---|---|---|---|---|
| No. | Alite | Belite | aluminate | aluminoferrite |
| M1 | 65.74 | 18.62 | 3.68 | 11.05 |
| M2 | 58.43 | 24.79 | 9.88 | 6.36 |
| M3 | 65.54 | 20.54 | 4.67 | 11.67 |

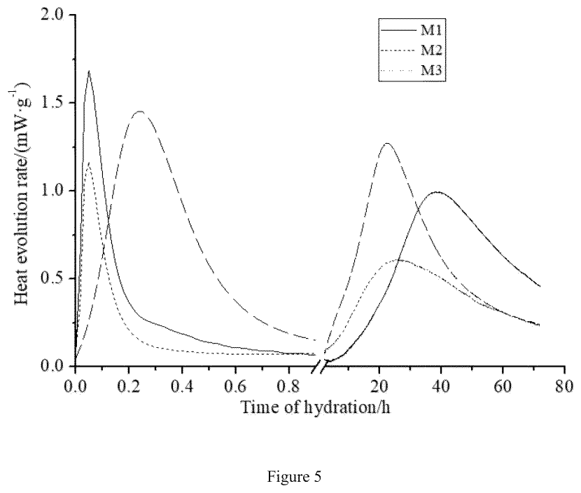

The hydration heat release test was conducted on three groups of cement clinker samples prepared, and the results are shown in FIG. 5. As can be seen, the three groups of cement clinker samples all show typical hydration and heat release curve characteristics of cement clinker. However, due to the differences in mineral composition, there are significant differences in the hydration exothermic peak shapes (rates) of the three groups of samples. The mineral composition of M1 and M3 is relatively similar, and the hydration exothermic peak in the early stage and main hydration exothermic peak shapes are also relatively similar. When compared to M3, the content of alite in M1 sample is relatively high, and the hydration exothermic peak in the early stage and main hydration exothermic peak are relatively strong, showing a relatively high hydration activity. The content of tricalcium aluminate in M2 sample is significantly high, and its hydration exothermic peak in the early stage is significantly strong. It can be seen that the hydration heat release characteristics and laws of the three groups of samples tally with the research results from classical literature. This indicates that the clinker samples prepared by the above-mentioned high-throughput experimental method can scientifically characterize the evolution law of basic physical and chemical properties appropriate to their composition, and can be used for the research of screening and optimization of the physical and chemical properties of cement-based materials.

### Embodiment 2

The target of embodiment 2 is to prepare a new type of low-carbon cement clinker. The system of the cement clinker to be prepared is a ye'elimite-ternesite cement clinker system, and the design proportion is shown in Table 3. Three types of cement clinker single-mines, namely ye'elimite, ternesite and dicalcium silicate, are prepared by calcination respectively according to the composition of cement clinker. Wherein, ternesite is a newly discovered low-carbon clinker mineral with hydration activity. Three types of clinker phases are ground into powder with a specific surface area of about 300±20 m²/kg. The above-mentioned single-mine raw materials are placed in the corresponding unit material storage tubes X1, X2, and X3 of the high-throughput test apparatus (see FIG. 1) respectively, wherein mixed zircon balls are added to X4. The raw material storage tube is placed into raw material devices in a circular arrangement. The raw material proportion of the cement clinker prepared is given according to Table 3, and the weight proportion of the raw material is controlled by controlling the valve size of the storage pipe orifice through the feeding control device. Different types and weights of raw materials are placed into different target material pre-loaded tanks through 360-degree circularly rotating the raw material device. 200 g zircon balls are added to the pre-loaded tank for each. The pre-loaded tank is placed into the mixing device. After the target clinker is evenly mixed in the pre-loaded tank, the zircon balls are separated through a mesh filter screen. The d cement clinker enters the target material terminal storage tanks Z1, Z2, and Z3 respectively, and the storage tanks are hermetically stored to obtain three groups of clinker samples, S1, S2, and S3.

TABLE 3
Composition of designed clinker (mass percentage/%)

| | | | Dicalcium |
|---|---|---|---|
| No. | Ye'elimitee | Ternesite | silicate |
| S1 | 30 | 50 | 20 |
| S2 | 30 | 30 | 40 |
| S3 | 30 | 10 | 60 |

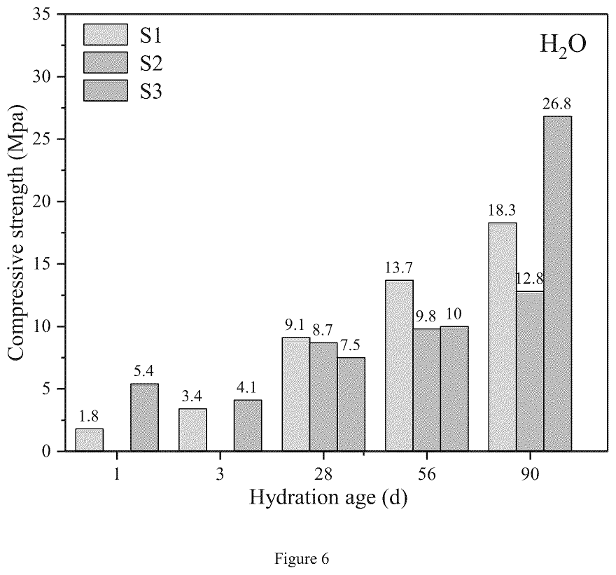

Three groups of cement clinker S1, S2, and S3 are mixed at the 0.4 w/c (a ratio of water and cement). The mixed slurry is poured into a mold with 30 mm×30 mm×30 mm, placed under standard constant temperature (20±1° C.) and humidity (95±1%) for 7 days. After hardening, the samples are demoulded and cured in water for 28, 56, and 90 days, respectively. The compressive strengths of them are measured, and the hydration synergism of three clinker minerals in a new system is analyzed. FIG. 6 shows the compressive strength results of S1, S2, and S3 paste samples at different curing ages. It can be seen that the compressive strength of S3 sample is the highest in the first three days of hydration, with 5.4 MPa and 26.8 MPa respectively. At the age of 28 to 56 days, the strength of S1 sample is the highest, with 9.1 MPa and 13.7 MPa, respectively. This indicates that a higher content of ternesite is beneficial for promoting the strength development of the clinker from 28 to 56 days. High content of dicalcium silicate can promote the strength development in the age of first 3 days and at the age from 56 to 90 days. S2 sample has the lowest strength at 90 days, only 12.8 MPa, and has almost no strength at ages of 1 day and 3 days. The strength at ages from 28 to 56 days is also lower. This indicates that the ratio of S2 clinker is not conducive to the synergistic hydration of ternesite and ye'elimite. This again indicates that the clinker samples prepared by the above-mentioned high-throughput experimental method can be used for screening and optimizing the composition, structure, and performance of cement-based materials.

### Embodiment 3

The target of embodiment 3 is to prepare cement, and the composition design of the target cement to be prepared is shown in Table 4. According to the clinker components involved in cement, four single-mines raw materials of clinker, namely alite (solid solution of tricalcium silicate), belite (solid solution of dicalcium silicate), tricalcium aluminate, and tetracalcium aluminoferrite, are prepared by calcination respectively. Raw materials for the structural unit of three types of cement from industrial sources, namely gypsum, slag, fly ash and limestone, are collected and prepared. The eight raw materials shall be processed and controlled to a certain particle size or fineness according to the fineness requirements of the target material, wherein the mineral surface area of the cement clinker is 300±20 m²/kg. Slag is processed into two particle size gradations respectively: slag 1 # has a specific surface area of 450±20 m²/kg, and slag 2 # has a specific surface area of 500±20 m²/kg. The specific surface area of fly ash is 450±20 m²/kg, and the specific surface area of limestone powder is 300±20 m²/kg. The above-mentioned unit raw materials are placed in the corresponding unit material storage tanks X1, X2, X3, X4, X5, X6, X7, X8, and X9 of the high-throughput test device (see FIG. 1). Zircon balls are added to X10. The raw material storage tubes are placed into a raw material device in a circular arrangement. The raw materials proportion of cement to be prepared is given according to table 1, and the weight proportion of raw materials is controlled according to the valve size of the storage tank orifice through the feeding control device. Different types and weights of raw materials are placed into different target material pre-loaded tanks Y1, Y2, Y3, Y4, Y5, Y6, Y7 and Y8 through 360-degree circularly rotating the raw material device. 200 g zircon balls are added to the pre-loaded tank for each. The pre-loaded tank is placed into the mixing device. After the target clinker is uniformly mixed in the pre-loaded tank, the zircon balls are separated through a mesh filter screen. The well mixed cement clinker enters the target material terminal storage tanks Z1, Z2, Z3, Z4, Z5, Z6, Z7 and Z8 respectively, and the storage tanks are hermetically stored to obtain eight groups of clinker samples, namely W1, W2, W3, W4, W5, W6, W7 and W8 respectively. At the same time, traditional process sample preparation method is adopted for the design composition of control samples W1 and W2. The designed cement clinker samples are first calcined and prepared, and then added with 5% and 15% slag, respectively. Finally, the control samples W1x and W2x are prepared by mixed grinding (see Table 5).

TABLE 4
Composition of cement designed in embodiment 3 (mass percentage/%)

| | | | Tricalcium | Tetracalcium | | Slag | Slag | fly | |
|---|---|---|---|---|---|---|---|---|---|
| No. | Alite | Belite | aluminate | aluminoferrite | Gypsum | 1# | 2# | ash | Limestone |
| W1 | 60 | 19 | 3 | 11 | 2 | 5 | | 0 | 0 |
| W2 | 50 | 21 | 6 | 6 | 2 | 10 | 5 | 0 | 0 |
| W3 | 60 | 19 | 3 | 11 | 2 | 0 | | 5 | 0 |
| W4 | 50 | 21 | 6 | 6 | 2 | 0 | | 15 | 0 |
| W5 | 50 | 21 | 8 | 6 | 5 | | 5 | 5 | 0 |
| W6 | 50 | 21 | 6 | 6 | 2 | | 5 | 5 | 5 |
| W7 | 50 | 21 | 8 | 6 | 5 | | 0 | 0 | 10 |
| W8 | 30 | 21 | 8 | 6 | 2 | | 5 | 5 | 3 |

TABLE 5
Designed Composition of cement of control samples (mass percentage/%)

| | | | Tricalcium | Tetracalcium | | | fly | |
|---|---|---|---|---|---|---|---|---|
| No. | Alite | Belite | aluminate | aluminoferrite | Gypsum | Slag | ash | Limestone |
| Control | 60 | 19 | 3 | 11 | 2 | 5 | 0 | 0 |
| sample | | | | | | | | |
| W1x | | | | | | | | |
| Control | 50 | 21 | 6 | 6 | 2 | 15 | 0 | 0 |
| sample | | | | | | | | |
| W2x | | | | | | | | |

According to GB/T12960 “Quantitative Determination of Constituents of Cement”, the contents of slag and fly ash in the above ten groups of cement prepared are measured and analyzed respectively. In this method, the selected dissolution method is used for samples containing fly ash, and the cement sample is selectively dissolved with the nitric acid solution, in which the component of the fly ash is insoluble while the other components are dissolved. The selective dissolution method is adopted for samples containing slag. After the cement sample is selectively dissolved by a solution containing EDTA at pH=11.60, the clinker minerals, gypsum, and carbonates are dissolved, while the other components are essentially insoluble. The content of limestone depends on the content of carbon dioxide. The carbon dioxide is determined by the alkali asbestos absorption weighing method. The content of each component in the cement is calculated from the results of selective dissolution, the content of carbon dioxide and sulphate sulfur trioxide in the cement. The determination results of gypsum, slag, fly ash, and limestone content in 10 groups of cement samples are shown in Table 6. It can be seen from the table that the determination results of gypsum, slag, fly ash and limestone content in 8 groups of cement samples prepared according to the method of the present application are tallying with the designed composition of cement. This indicates that 8 groups of cement samples with set composition have been successfully prepared according to the method of the present application. However, the comparison of W1x and W2x prepared by traditional test methods has a relatively large error in slag content compared to the actual mixing amount. This is mainly due to the traditional process, in which clinker is prepared by calcining first and then adding admixtures to prepare cement adopted by the control sample. This complex sample preparation process o leads to certain sample composition design errors. When compared to the method of the present application, the clinker prepared by calcination not only has the component solid solution and vitreous phase but also has problems of insufficient reaction kinetics and non-ideal chemical equilibrium state, resulting in the presence of unreacted phases or components such as free calcium oxide. Among them, the dissolution characteristics of the vitreous phase are similar to those of slag. The presence of the vitreous phase cannot be effectively distinguished when using selectively dissolved method. It can be seen that the sample prepared by the present application is closer to the actual theoretical composition of the sample, which is conducive to in-depth revealing the changes in the composition and structure of the sample.

TABLE 6
Composition of determined cement (mass percentage/%)

| | | | | | fly | | | |
|---|---|---|---|---|---|---|---|---|
| No. | Gypsum | Error | Slag | Error | ash | Error | Limestone | Error |
| W1 | 2.1 | 0.1 | 5.2 | 0.2 | 0.0 | 0.0 | 0.00 | 0.0 |
| W2 | 2.0 | 0.0 | 14.9 | −0.1 | 0.0 | 0.0 | 0.00 | 0.0 |
| W3 | 1.9 | −0.1 | 0.0 | 0.0 | 4.7 | −0.3 | 0.00 | 0.0 |
| W4 | 2.1 | 0.1 | 0.0 | 0.0 | 14.5 | −0.5 | 0.00 | 0.0 |
| W5 | 4.9 | −0.1 | 4.7 | −0.3 | 4.8 | −0.2 | 0.00 | 0.0 |
| W6 | 1.9 | −0.1 | 4.6 | −0.4 | 5.1 | 0.1 | 5.02 | 0.02 |
| W7 | 5.1 | 0.1 | 0.0 | 0.0 | 0.0 | 0.0 | 10.01 | 0.01 |
| W8 | 2.2 | 0.2 | 4.9 | −0.1 | 4.8 | −0.2 | 3.05 | 0.05 |
| Control | | | | | | | | |
| sample | 0.0 | 0.0 | 5.7 | 0.7 | 0.0 | 0.0 | 0.00 | 0.0 |
| W1x | | | | | | | | |
| Control | 0.0 | 0.0 | 15.8 | 0.8 | 0.0 | 0.0 | 0.0 | 0.0 |
| sample | | | | | | | | |
| W2x | | | | | | | | |

Ten groups of cement samples are moulded and prepared in accordance with GB/T17671 “Test method for strength of hydraulic cement mortar”, and their strength properties at 3 and 28 days are measured. The results are shown in Table 7. It can be seen from the table that different cement samples prepared by the present application method exhibit significant differences in strength properties due to differences in composition and fineness, wherein cement with relatively high alite content or a certain amount of gypsum and slag, especially samples with finer slag fineness, exhibit relatively excellent properties. The W-5 sample has excellent flexural and compressive strength simultaneously. It can be seen that this present application method can be used to quickly prefer and optimize the composition and fineness or gradation of the designed cement. In addition, it is not difficult to see from the comparison that W1x and W2x prepared by the traditional process and the theoretical design possess certain differences in their performance due to the relatively large deviation between the composition of the control samples when compared to the W1 and W2 samples prepared by the method of the present application. As mentioned earlier, this is mainly due to the large difference between the actual composition and theoretical actual composition of the samples prepared by using traditional methods. In traditional sample preparation methods, although the corresponding composition is designed according to the idealization during the calcination process, it is often inevitable to form a vitreous phase, solid solutions of components, unreacted phase components and so on during the actual firing process.

Obviously, the performance of W1 and W2 samples prepared by the method of the present application is closer to the performance of the theoretically designed sample composition. This indicates that this method can reduce sample preparation errors in complex processes, and using structural unit materials such as single-mine or single-phase as basic elements can directly match and design the mineral phase composition of cement. That is more consistent with the original design intent, conducive to mastering more objective and realistic the changing law in material composition and structure. That is also beneficial to essentially reveal key factors, conditions, and laws that affect material performance, and promote scientific and effective screening of material components and properties with excellent performance. At the same time, the development of various new cement-based materials can be accelerated based on more objective and scientific change laws in material composition and structure.

TABLE 7
Strength properties of prepared cement samples

| | Flexural | Compressive | Flexural | Compressive |
|---|---|---|---|---|
| | strength | strength | strength | strength |
| | at 3 days | at 3 days | at 28 | at 28 |
| No. | (Mpa) | (Mpa) | days (Mpa) | days (Mpa) |
| W1 | 6.2 | 29.8 | 7.3 | 51.9 |
| W2 | 5.3 | 28.2 | 6.8 | 48.7 |
| W3 | 5.9 | 28.7 | 8.3 | 49.9 |
| W4 | 5.2 | 28.2 | 6.5 | 49.5 |
| W5 | 5.8 | 29.5 | 8.3 | 51.4 |
| W6 | 4.9 | 26.2 | 6.3 | 45.2 |
| W7 | 5.1 | 27.1 | 6.7 | 47.7 |
| W8 | 6.1 | 30.2 | 8.5 | 51.5 |
| Control | 6.5 | 30.1 | 7.8 | 53.2 |
| sample W1x | | | | |
| Control | 5.7 | 28.8 | 7.1 | 49.5 |
| sample W2x | | | | |

In addition, fineness and gradation have a significant impact on the performance of cement materials and concrete. In the preceding embodiments, the fineness parameters of the corresponding clinker single-mine are recorded, and there are two types of records for slag with different fineness. However, in practical applications, it is necessary to control the fineness of the clinker and slag according to the design requirements, such as grinding the clinker single-mine into a powder with a specific surface area of about 300±20 m²/kg; slag can be designed in different fineness or gradation such as 300 m²/kg, 500 m²/kg, 700 m²/kg, etc. When preparing concrete, a person skilled in this field reasonably configures various gradation fineness settings such as sand and clinker based on the technical content disclosed in the present application.

The present application is not limited to the above-mentioned embodiments. Based on the technical solution disclosed in the present application, a person skilled in this field can make some substitutions and modifications to some of the technical features without creative labor based on the disclosed technical content. These substitutions and modifications are within the protection scope of the present application.

1. A method for high-throughput preparation of a cement-based material,
wherein single-mine, single-phase, or a unit component maintaining specific hydration and hardening characteristics are used as a structural unit,
the method comprises following steps:
1) placing the structural units in storage tubes X1, X2, X3 . . . Xn respectively;
2) putting the materials in each of the storage tubes according to the design proportion of the cement-based materials, and then preparing Y1, Y2, Y3 . . . Ym of mixed materials respectively;
3) fully and uniformly mixing the Y1, Y2, Y3 . . . Ym of the mixed materials through a uniform mixing device respectively; and
4) filling the Y1, Y2, Y3 . . . Ym of the uniformly mixed materials into storage tanks Z1, Z2, Z3 . . . Zm respectively to prepare m groups of cement-based material samples.

2. The method for high-throughput preparation of a cement-based material according to claim 1, characterized in that the structural unit is a single-mine of cement clinker which comprises alite (a solid solution of tricalcium silicate C₃S), belite (a solid solution of dicalcium silicate C₂S), tricalcium aluminate (C₃A), calcium aluminate (CA), calcium dialuminate (CA₂), mayenite (C₁₂A₇), dicalcium ferrite (C₂F), tetracalcium aluminoferrite (C₄AF), hexacalcium aluminodiferrite (C₆AF₂), hexacalcium dialuminoferrite (C₆A₂F), ye'elimite (C₄A₃$), calcium fluoaluminate (C₁₁A₇·CaF₂), calcium chloroaluminate (C₁₁A₇·CaCl₂), barium calcium sulphoaluminate (C₃A₃$·BaO), strontium calcium aluminate (C₃A₃$·SrO), ternesite (C₅S₂$), tricalcium phosphate (C₃P), tetracalcium phosphate (C₄P), calcium phosphoaluminate (C₈A₄P), gehlenite (C₂AS), periclase (MgO), free gypsum (f-CaSO₄), and free calcium oxide (f-CaO).

3. The method for high-throughput preparation of a cement-based material according to claim 2, wherein
the structural unit further comprises a cement component unit including gypsum, slag, volcanic ash, fly ash, silica fume, and limestone.

4. The method for high-throughput preparation of a cement-based material according to claim 2, wherein
the structural unit further comprises a concrete structural unit including sand, stone, and concrete admixtures, as well as supplementary cementitious materials such as phosphorus slag, steel slag and so on.

5. The method for high-throughput preparation of a cement-based material according to claim 2, wherein
the structural unit further comprises a single-mine of cement clinker, cement components, supplementary cementitious materials, and sand/gravel materials with different fineness and particle gradation.

6. The method for high-throughput preparation of a cement-based material according to claim 1, wherein
performing composition structure and performance tests on the m groups of cement-based material samples respectively for screening specific or excellent performance cement-based materials and producing data, the composition structure and performance tests, including chemical composition, mineral composition, density, fineness, hydration heat evolution, setting time, strength, composition and structure of hydration products, volume soundness, impermeability, freeze-thaw resistance, workability, sulfate resistant, chloride resistant, and wear resistance.

7. An apparatus for realizing the method for high-throughput preparation of a cement-based material according to claim 1, comprising:
n of primary storage silos for accommodating raw materials of cement-based material composition unit, wherein flow valves are provided at bottom ends of each of the primary storage silos, and
m of secondary pre-loaded tanks for receiving materials from any two or more of n of the primary storage silos and mixing the materials uniformly;
wherein n of the primary storage silos are arranged on a primary storage silo delivery device in a rotating or linear sliding manner, and m of the secondary pre-loaded tanks are arranged on a secondary pre-loaded tank delivery device in a rotating or linear sliding manner;
a feeding control device is provided between the primary storage silo delivery device and the secondary pre-loaded tank delivery device.

8. The apparatus for realizing the method for high-throughput preparation of a cement-based material according to claim 7, further comprising
m of terminal storage tanks for receiving target material discharged from the secondary pre-loaded tanks.
